(19) United States
(12) Patent Application Publication
(10) Pub. No.: US 2021/0181528 A1
(43) Pub. Date: Jun. 17, 2021

(54) DEVICES AND METHODS FOR CORRECTING HIGH-ORDER OPTICAL ABERRATIONS FOR AN EYE USING LIGHT

(71) Applicant: Ovitz Corporation, Rochester, NY (US)

(72) Inventors: Joung Yoon KIM, Seoul (KR); Nicolas Scott BROWN, Rochester, NY (US)

(21) Appl. No.: 17/109,928

(22) Filed: Dec. 2, 2020

Related U.S. Application Data
(60) Provisional application No. 62/948,122, filed on Dec. 13, 2019.

(51) Int. Cl. G02C 7/02; A61F 2/16; A61F 9/008

(52) U.S. Cl. CPC G02C 7/02; A61F 2/16; G02C 2202/24; G02C 2202/20; A61F 9/00804

(57) ABSTRACT

Devices and methods for correcting and compensating high order aberrations from an eye using light are described. A method includes providing a first light to a first region of an optical element, thereby modifying a refractive index profile in the first region of the optical element, while offsetting a center of the first region with respect to a center of the optical element. The refractive index profile is configured to compensate for an optical aberration of a subject's eye.

### RELATED APPLICATIONS

This application claims the benefit of, and priority to, U.S. Provisional Patent Application No. 62/948,122, filed Dec. 13, 2019, which is hereby incorporated by reference in its entirety.

### TECHNICAL FIELD

This application relates generally to correcting high-order optical aberrations for an eye using light.

### BACKGROUND

Eyes are important organs, which play a critical role in human's visual perception. An eye has a roughly spherical shape and includes multiple elements, such as cornea, lens, vitreous humour, and retina. Imperfections in these components can cause reduction or loss of vision. For example, too much or too little optical power in the eye can lead to blurring of the vision (e.g., near-sightedness or far-sightedness), and astigmatism can also cause blurring of the vision.

Corrective lenses (e.g., glasses and contact lenses) are frequently used to compensate for blurring caused by too much or too little optical power and/or astigmatism. However, when eyes have higher order aberrations (e.g., aberrations higher than astigmatism in the Zernike polynomial model of aberrations), conventional corrective lenses cannot adequately compensate for all of the aberrations associated with the eyes, resulting in blurry images even when corrective lenses are used.

### SUMMARY

Accordingly, there is a need for corrective lenses or corrective procedures that can compensate for higher order aberrations and methods and devices that can accurately measure higher order aberrations so that proper corrective lenses can be designed and/or made, and corrective procedures performed.

The above deficiencies and other problems associated with conventional devices and corresponding methods are reduced or eliminated by the disclosed devices and methods.

As described in more detail below, some embodiments involve a method that includes providing a first light to a first region of an optical element, thereby modifying a refractive index profile in the first region of the optical element, while offsetting a center of the first region with respect to a center of the optical element. The refractive index profile is configured to compensate for an optical aberration of a subject's eye.

In accordance to some embodiments, a method includes providing a first light to a first region of a biological tissue in a subject's eye, thereby modifying a refractive index profile in the first region of the biological tissue, while offsetting a center of the first region with respect to a center of a pupil of the subject's eye. The refractive index profile is configured to correct an optical aberration of the subject's eye.

In accordance to some embodiments, a method of improving vision of a subject includes placing an optical element having a vision correction profile in an optical path of light that impinges on a retina of the subject. A center of the vision correction profile is offset from a center of the optical element.

In accordance to some embodiments, a method includes improving vision of a subject by placing an optical element having a vision correction profile in an optical path of light that impinges on a retina of the subject. A center of the vision correction profile is offset from a center of the optical element.

Also described is a lens that includes a substrate with a refractive index profile, the refractive index profile having a first refractive index at a first location of the substrate, and a second refractive index, different from the first refractive index, at a second location of the substrate, wherein a center of the refractive index profile is offset from a center of the lens.

In accordance with some embodiments, a corrective lens (e.g., a contact lens) is made by any method described herein.

Thus, devices are provided with more efficient and accurate methods for performing wavefront sensing and determining vertices of contact lenses, thereby increasing the effectiveness, efficiency, accuracy, and user satisfaction with such devices. Such devices and corresponding methods may complement or replace conventional methods for performing wavefront sensing.

### BRIEF DESCRIPTION OF THE DRAWINGS

For a better understanding of the various described embodiments, reference should be made to the Description of Embodiments below, in conjunction with the following drawings in which like reference numerals refer to corresponding parts throughout the figures.

FIGS. 1A-1D are schematic diagrams illustrating compensation of higher order aberrations in accordance with some embodiments.

FIG. 1E is an image of an eye that illustrates offset between an optical reference axis of an eye and a center of a contact lens in accordance with some embodiments.

FIGS. 2A-2D illustrate results of a clinical study showing the benefit of a contact lens with a center-offset surface profile to compensate for the higher order aberrations.

FIG. 3 is a schematic diagram of a system 300 used to form a refractive index profile in a lens to compensate for optical aberrations in accordance with some embodiments.

FIG. 4 is a schematic diagram of a system used to correct an optical aberration in a subject's eye, in accordance with some embodiments.

FIG. 5 is a schematic diagram illustrating a refractive index profile of a lens in accordance with some embodiments.

### DESCRIPTION OF EMBODIMENTS

Reference will be made to embodiments, examples of which are illustrated in the accompanying drawings. In the following description, numerous specific details are set forth in order to provide a thorough understanding of the various described embodiments. However, it will be apparent to one of ordinary skill in the art that the various described embodiments may be practiced without these particular details. In other instances, methods, procedures, components, circuits, and networks that are well-known to those of ordinary skill in the art are not described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first image sensor could be termed a second image sensor, and, similarly, a second image sensor could be termed a first image sensor, without departing from the scope of the various described embodiments. The first image sensor and the second image sensor are both image sensors, but they are not the same image sensor.

The terminology used in the description of the embodiments herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

Optical aberrations in human eyes are quite common. Thus, optical devices (e.g., glasses or contact lenses) designed to compensate for lower order aberrations (e.g., defocus, such as myopia or hyperopia, and astigmatism) are widely used. However, correction of, or compensation for, higher order aberrations (e.g., aberrations higher than astigmatism in the Zernike polynomial model of aberrations, such as trefoil, coma, quadrafoil, spherical aberration, and secondary astigmatism) has remained more challenging.

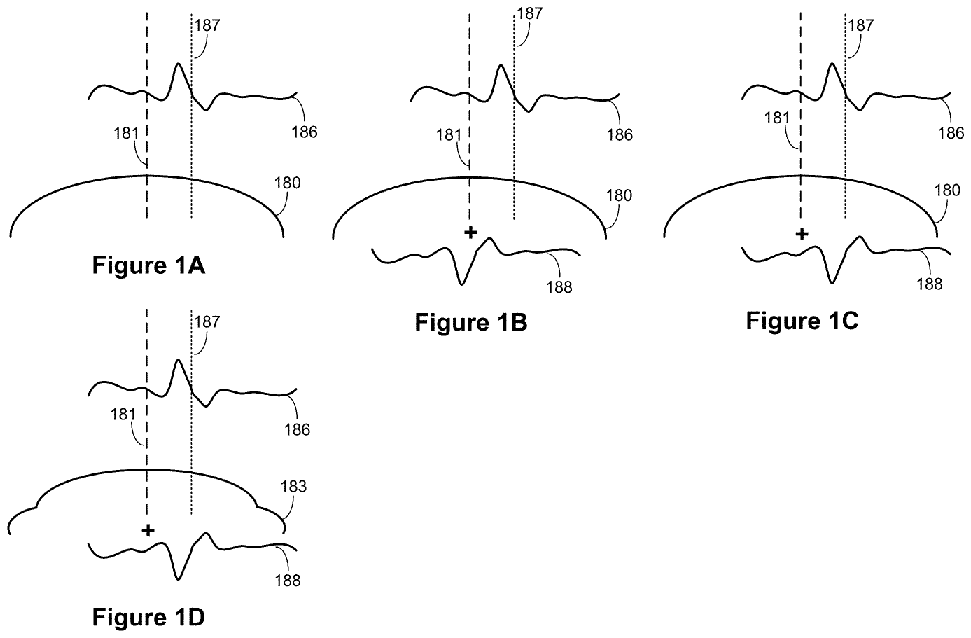

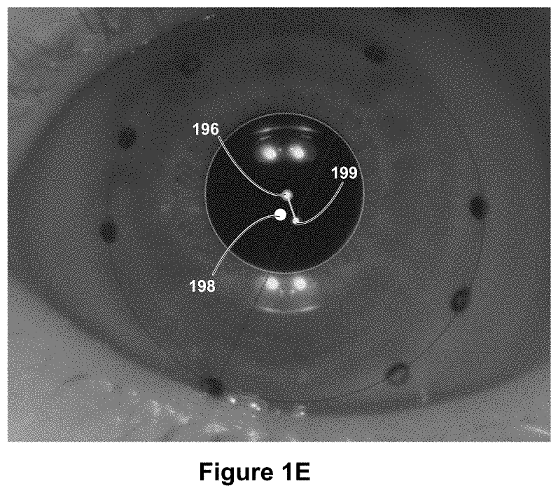

FIGS. 1A-1D are schematic diagrams illustrating compensation of higher order aberrations in accordance with some embodiments.

FIG. 1A illustrates higher order aberrations of an eye, represented by line 186, and a surface profile of a contact lens 180 without high-order correction. As a result, an eye wearing the contact lens 180 may see the higher order aberrations, even after defocusing (e.g., myopia or hyperopia) and astigmatism are compensated for by the contact lens 180. FIG. 1A also illustrates that the visual axis 187 of the eye is typically not aligned with the centerline 181 of the contact lens 180. Thus, the higher order aberrations measured from an eye (which are aligned with, for example, the visual axis 187 of the eye) are not aligned with the centerline 181 of the contact lens 180.

FIG. 1B illustrates modification of the surface profile of the contact lens 180 by superposing a surface profile 188 configured to compensate for the higher order aberrations. However, when the surface profile 188 is positioned around the centerline 181 of the contact lens 180 as shown in FIG. 1B, the combined surface profile is not effective in reducing the higher order aberrations, as the surface profile 188 is offset from the higher order aberrations measured along the visual axis 187 of the eye.

FIG. 1C illustrates modification of the surface profile of the contact lens 180 by superposing the surface profile 188 configured to compensate for the higher order aberrations where the surface profile 188 is positioned around the visual axis 187 of the eye instead of the centerline 181 of the contact lens 180.

FIG. 1D is similar to FIG. 1C except that the modification of the surface profile can be applied to a multifocal lens 183.

FIG. 1E illustrates offset between an optical reference axis (e.g., an axis based on a corneal vertex) of an eye and a center (or a vertex) of a contact lens in accordance with some embodiments. The contact lens shown in FIG. 1E has markers around its periphery, and based on positions of the markers, a center 199 of the contact lens can be determined. In some embodiments, the corneal vertex 198 is determined based on subject-fixated coaxially sighted light reflex. In some embodiments, the subject-fixated coaxially sighted light reflex is a subject-fixated coaxially sighted corneal light reflex (e.g., light reflected by a corneal surface, such as an anterior corneal surface) or a subject-fixated coaxially sighted contact lens light reflex (e.g., light reflected by a surface of a contact lens, such as an anterior contact lens surface). Based on a pattern of light illuminating the eye, a corneal vertex 198 of the eye can be determined. By utilizing such information, a surface profile for correcting high-order aberrations can be placed based on the location of the corneal vertex 198 of the eye.

FIG. 1E also illustrates offset between a center 196 of the pupil from the corneal vertex 198 of the eye and the center 199 of the contact lens. A contact lens for which the surface profile for correcting high-order aberrations is positioned based on the location of the center 196 of the pupil is less effective in correcting high-order aberrations.

### Correcting Higher-Order Aberrations Improves Vision

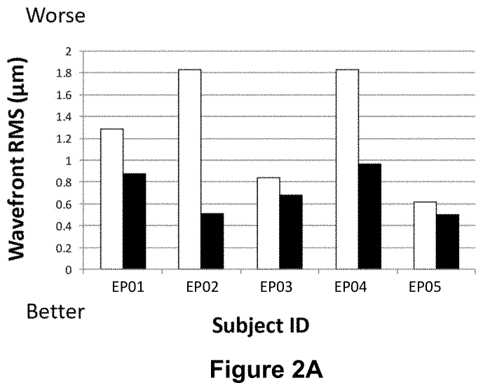

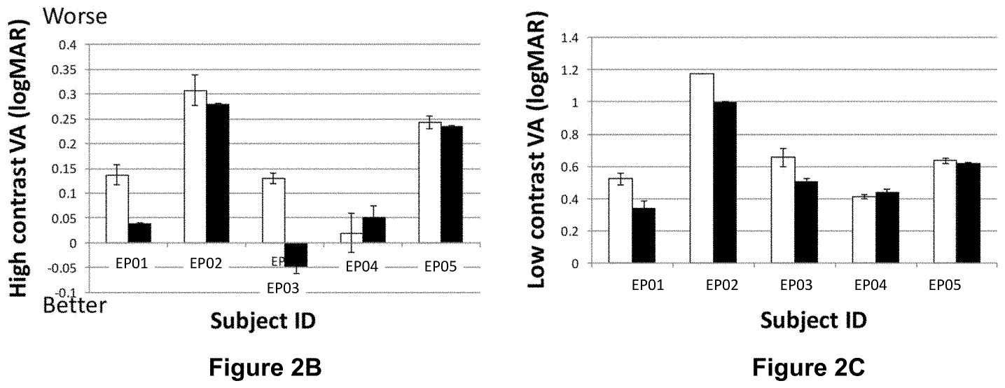

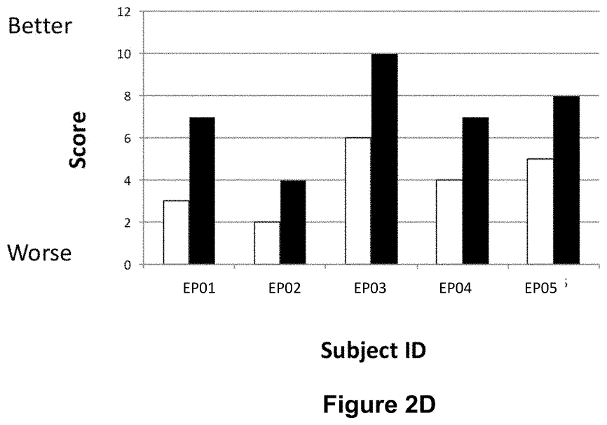

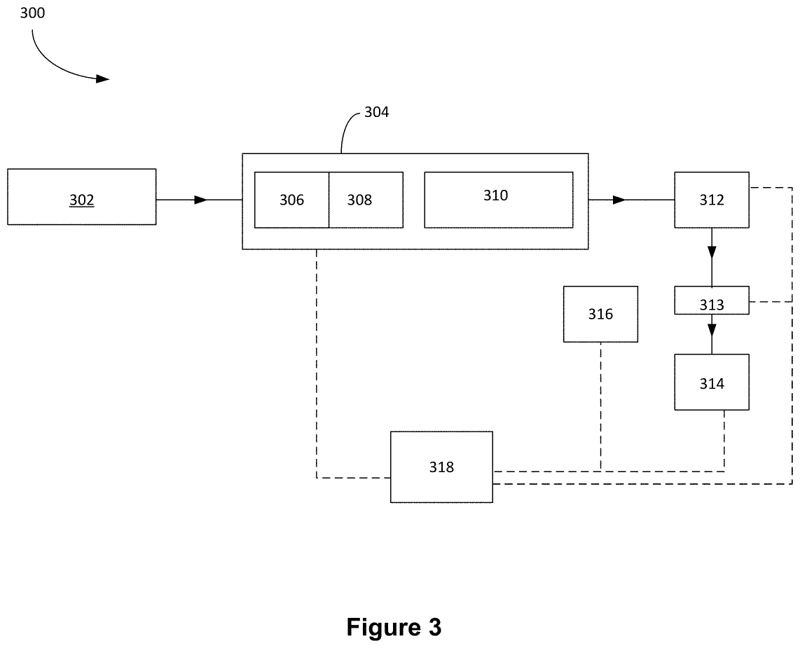

FIGS. 2A-2D illustrate results of a clinical study showing the benefit of a contact lens (e.g., a scleral lens) with a center-offset surface profile to compensate for the higher order aberrations.

Clinical studies were conducted on patients with keratoconus (KCN) who had already fitted scleral lenses in both eyes. One eye of each patient, which required overrefraction (e.g., sphere and cylinder correction), or pinhole or surface eccentricity to see better, was studied. Visual performance measurements were performed with overrefraction.

Baseline wavefront measurement was done with cycloplegia while measurements involving higher order aberration Prosthetic Replacement of Ocular Surface Ecosystem (HOA PROSE) was done with natural dilation (FIG. 2A, in which the wavefront root-mean-squared (RMS) error for a spherical Prosthetic Replacement of Ocular Surface Ecosystem without a surface profile to compensate for higher order aberrations is shown in white and the wavefront RMS error for the HOA PROSE is shown in black). As shown in FIG. 2A, the higher order wavefront RMS error was reduced for all subjects by using the HOA PROSE instead of a conventional PROSE.

Additional objective outcome measures include both high contrast visual acuity (VA) (shown in FIG. 2B) and low contrast VA (shown in FIG. 2C). As shown in FIGS. 2B and 2C, both high contrast VA and low contrast VA were improved with the HOA PROSE in 4 out of 5 subjects.

Subjective outcomes were also measured by the subject scoring his quality of vision (e.g., from 0—totally blurred, 5—somewhat blurred, to 10—perfectly sharp), and the results are shown in FIG. 2D. As shown in FIG. 2D, subjective visual quality was improved in all 5 out of 5 subjects.

These results show the improved vision for using the HOA PROSE (having the offset vision correction profile) for patients with KCN, as measured by both objective visual acuity and subjective preference. In addition, the reduced higher order wavefront root mean square aberrations obtained with the HOA PROSE correlate well with visual performance.

In some embodiments, a contact lens with the superposed surface profile (e.g., a superposition of the surface profile 188 and a profile of the contact lens 180 or the multifocal contact lens 183, such as the HOA PROSE) is made by machining (e.g., using a computer numerical control machine). For example, the lens is cut from a lens material (e.g., polymethyl methacrylate, silicone acrylate, fluorocarbon acrylate, fluorocarbon sulfone, hexafocon, enfulfocon, oprifocon, itafluorofocon, etc.) to have the selected surface profile. In some embodiments, the lens is made by three-dimensional printing to have the superposed surface profile.

In some embodiments, the contact lens 180 or the multifocal contact lens 183 is further modified by Intra-Polymer Refractive Index Shaping (IRIS) described below to have the effect of the superposed surface profile. For example, IRIS causes light-induced changes in optical polymers, such as intraocular lenses, contact lenses or corneal inlays. In some embodiments, the contact lens with the superposed surface profile is further modified by IRIS. Such further modification may be used to add additional surface profile (e.g., a surface profile for correcting even higher order aberrations, a surface profile for providing multiple foci, etc.) or correct any errors induced by machining or any variation in the lens material across the lens.

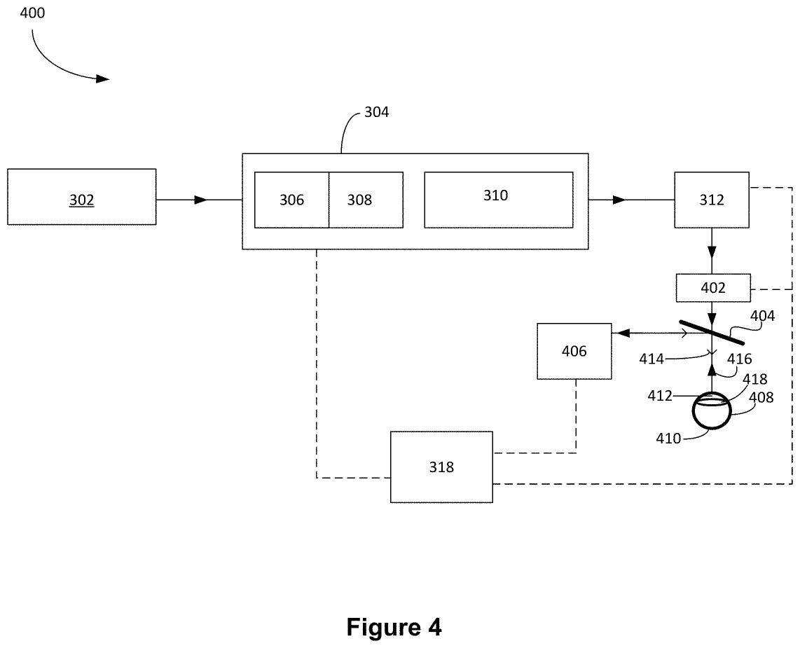

FIG. 3 is a schematic diagram of a system 300 used to form a refractive index profile in a lens to compensate for optical aberrations in accordance with some embodiments. The system 300 includes a laser 302. An output of the laser is sent to a beam conditioning unit 304. In some embodiments, the beam conditioning unit 304 includes a temporal conditioning unit 306, a temporal pulse measurement unit 308, and a spatial conditioning unit 310. An output of the beam conditioning unit 304 is sent to a shutter 312, which physically blocks a beam from interacting with a sample placed in a sample manipulation unit 314. Laser light that is not blocked by the shutter 312 passes through optics 313 before impinging on the sample. In some embodiments, the optics 313 includes an objective lens. In some embodiments, a monitoring unit 316 monitors the process occurring at the sample. Dotted lines in FIG. 3 denote electrical and/or signal connections between a computer 318 and one or more of: the beam conditioning unit 304, the shutter 312, the optics 313, the sample manipulation unit 314, and the monitoring unit 316.

In some embodiments, the laser 302 includes a pulse laser, such as a Kerr-lens mode-locked Ti:Sapphire laser (e.g., Kapteyn-Murnane Labs, Boulder, Colo.) pumped by a pump laser (e.g., 4 W of a frequency-doubled Nd:YVO4 laser). In some embodiments, the laser 302 produces visible or near-IR light. In some embodiments, the laser pulses from the laser 302 have a wavelength between 600 and 1,000 nm. In some embodiments, the laser pulses from the laser 302 have a wavelength between 700 and 900 nm. In some embodiments, the laser pulses from the laser 302 have a wavelength between 1,000 and 1,300 nm. In some embodiments, the laser pulses from the laser 302 have a wavelength between 350 and 600 nm. In some embodiments, the laser 302 is an amplified fiber laser emitting light with a wavelength from 1,000 nm to 1,600 nm.

In some embodiments, laser pulses from the laser 302 have a pulse energy from 0.01 nJ to 10 nJ. In some embodiments, the laser pulses have a pulse energy between 0.1 and 2 nJ. In some embodiments, an average power of the laser 302 is between 1 mW and 1,000 mW. In some embodiments, a repetition rate of the laser pulses of the laser 302 is between 1 MHz and 10 GHz. In some embodiments, the repetition rate is between 10 MHz and 500 MHz. In some embodiments, a pulse duration of the laser pulses emitted from the laser 302 is between 5 fs to 1 ps, for example, between 10 fs and 300 fs, between 30 to 200 fs. In some embodiments, laser 302 produces pulses of 300 mW average power, 30 fs pulse width, and 93 MHz repetition rate at a wavelength of 800 nm.

In some embodiments, less than 50% of the power from the laser 302 is delivered to the sample housed in the sample manipulation unit 314. For example, power loss, such as reflective power losses from optical elements (e.g., mirrors, prisms, one or more objective lens) lowers a measured average laser power at a focus of the optics 313 in the sample. In some embodiments, power losses lower the power delivered to the sample to about or less than 50% of the output power at the laser 302. In some embodiments, the average power at the focus of an objective of the optics 313 in the sample is about 120 mW, giving a pulse energy of about 1.3 nJ. In some embodiments, average laser power greater than 200 mW is obtained at the objective focus. In some embodiments, average laser power greater than 500 mW is obtained at the objective focus.

To ensure that the pulse peak power delivered to sample exceeds the nonlinear absorption threshold of the material, the pulse duration is compressed to the shortest possible duration (e.g., transform-limited pulse duration). In some embodiments, the transform-limited pulse is about 27 fs in duration. In some embodiments in which the optics 313 include a focusing objective lens, a large amount of glass is present inside the focusing objective and the positive dispersion caused by the glass increases the pulse duration. In some embodiments, the temporal conditioning unit 306 includes an extra-cavity compensation scheme to provide negative dispersion to compensate for the positive dispersion introduced by the focusing objective. In some embodiments, the temporal conditioning unit 306 includes two SF10 prisms and a retroreflector to form a two-pass, one-prism-pair configuration. In some embodiments, the two prisms are separated by 37.5 cm to compensate for the positive dispersion of the microscope objective and other optics within the optical path.

In some embodiment, the temporal pulse measurement unit 308 includes an autocorrelator (e.g., a collinear autocorrelator) to measure the pulse duration at the focus of the optics 313. In some embodiments, the autocorrelator uses third-order harmonic generation to measure the pulse duration. In some embodiments, second-harmonic generation is used to measure pulse duration in autocorrelation measurements. In some embodiments, these measurements are made for low numerical aperture (NA) objectives. In some embodiments, these measurements are made for high NA objectives.

In some embodiments, third-order surface harmonic generation (THG) is used to measure pulse width at the focus of the high-numerical aperture (NA) objectives based on autocorrelation. Such measurements have high signal to noise ratio, and do not introduce material dispersion that second harmonic generation (SHG) crystals can introduce. In some embodiments, a THG signal is generated at an interface of air and an ordinary cover slip (e.g., Corning No. 0211 Zinc Titania glass), and measured with a photomultiplier and a lock-in amplifier. In some embodiments, the optics 313 includes a 60×0.70 NA Olympus LUCPlanFLN long-working-distance objective.

In some embodiments, the laser beam from the laser 302 is spatially diverging. In some embodiments, the spatial conditioning unit 310 includes a concave mirror pair to adjust a dimension of the laser beam so that the laser beam can optimally fill an objective aperture in the optics 313.

In some embodiments, the sample manipulation unit 314 includes a translation stage that can move along three orthogonal axes (e.g., DC servo motor stage, a Newport VP-25XA linear stage having a resolution of 100 nm) and/or a translation stage that can move along two orthogonal axes (e.g., a 2D piezo nanopositioning stage, a PI P-622.2CD piezo stage having a resolution of 0.7 nm). The sample manipulation unit 314 is controlled and programmed by a computer 318 as a scanning platform to support and position one or more samples. In some embodiments, the sample includes a contact lens. In some embodiments, the sample includes an intraocular lens. In some embodiments, the sample includes a corneal inlay that has not been surgically implanted in the subject. In some embodiments, one or more translation stages in the sample manipulation unit 314 have a scanning speed between 0.1 micrometer/s and 100 mm/s. The scanning speed of the laser is preferably at least 0.4 μm/s, more preferably at least 0.1 mm/s, or at least 1 mm/s or at least 10 mm/s, and more preferably greater than 50 mm/s and higher. For example, scan speeds of 100 mm/s, 200 mm/s, 400 mm/s and up to 700 mm/s and even higher and all speeds in between are valuable. Such scanning speeds are effective in reducing treatment time. In some embodiments, for lasers emitting average laser powers greater than 200 mW, the scan rates may range above 10 mm/s, even as high as 500 mm/s or higher.

In some embodiments, one or more translation stages in the sample manipulation unit 314 scan a focus spot of the optics 313 (e.g., a focus of an objective lens) at a scanning speed of at least 1 mm/s. In some embodiments, the one or more translation stages in the sample manipulation unit 314 scan the focus spot at a scanning speed of at least 100 mm/s. In some embodiments, the translation stages include servo stages that have a DC servo-motor, allowing it to move smoothly between adjacent steps.

In some embodiments, a cross-sectional spot size of the focus spot is between 0.5 μm and 2 μm. In some embodiments, the range of spot size is between 0.5 μm to 2, 10, or 50 μm. In some embodiments, a spot length along an optical path of the laser beam (e.g., the z-axis, or depth along the axis of the laser beam) has similar dimensions as the cross-sectional spot size. In some embodiments, a spot length along an optical path of the laser beam (e.g., the z-axis, or depth along the axis of the laser beam) has different dimensions from the cross-sectional spot size. In some embodiments, the spot length along the z-axis is within a range between 1-50 μm, for example, between 1-20 μm.

In some embodiments, the laser 302 has a peak intensity at a focus of the sample of greater than 10¹³ W/cm². In some embodiments, the laser 302 has a peak intensity at a focus of the sample of greater than 10¹⁴ W/cm², or greater than 10¹⁵ W/cm². For peak intensities exceeding one terawatt per square centimeter, the possibility of two or more laser photons being simultaneously absorbed by the sample material becomes significant. In some embodiments, the amount of two-photon absorption is adjusted by doping or otherwise including in the irradiated material chromophores that exhibit large two-photon absorption cross-section at the proper wavelength (e.g., between 750 nm and 1100 nm). Absorption cross section is a measure for the probability of an absorption process. In some embodiments, the two-photon absorption cross-section is at least 10 GM. One GM is 10⁻⁵⁰ cm⁴ s photon⁻¹.

In some embodiments, the shutter 312 includes an optical shutter controlled by the computer with 1 ms time resolution. The shutter 312 allows the system 300 to precisely control the laser exposure time. In some embodiments, the shutter 312 and the sample manipulation unit 314 generate (e.g., micromachine) different patterns in the one or more samples using different scanning speeds at different position or depth in the sample. In some embodiments, the different patterns are generated using different laser exposure times.

In some embodiments, the monitoring unit 316 includes a CCD camera positioned close to the sample and a display to monitor the processes (e.g., micromachining) occurring at the sample in real time.

### Irradiating Polymers

A hydrogel is a three-dimensional (3D) network of hydrophilic polymers that can swell in water and hold a large amount of water while maintaining the structure due to chemical or physical cross-linking of individual polymer chains. In some embodiments, irradiated portions of the optical, hydrogel polymeric material exhibit a positive change in refractive index of about 0.01 or more. In some embodiments, the refractive index of the region increases by about 0.03 or more. In some embodiments, hydrated Akreos™ intraocular lens (IOL) material experiences an increase of refractive index of about 0.06. In some embodiments, using 1.3 nJ pulses, smooth lines 40 μm long are inscribed below a surface of a hydrogel, below the optical breakdown threshold of that material. The 1.3 nJ pulses may have a laser focal diameter of about 2.5 μm, giving rise to laser-irradiated lines about 1 μm wide and 3 μm deep. The lines change a refractive index of the hydrogel by 0.06.

In some embodiments, light reduces a refractive index of an irradiated region of the sample. In other words, the refractive index change is negative (e.g., a modified refractive index of the sample is lower than an original/native refractive index of the sample prior to radiation). In some embodiments, the magnitude of the negative change is about 0.01 or greater (e.g., −0.01 to −0.06 or more). In some embodiments, a negative change in refractive index of −0.03 or more is induced in a contact lens material. Further examples are disclosed in Femtosecond Laser Writing of Freeform Gradient Index Microlenses in Hydrogel-based Contact Lenses, by Gandara-Montano et al., in Optical Materials Express, vol. 5, no. 10, pp. 2257-2271 (2015), which is hereby incorporated by reference in its entirety.

IRIS does not change the Raman spectrum of hydrogels. Thus, without limiting the scope of claims, it is believed that laser irradiation only changes the refractive index of the hydrogel, but not its material composition or chemistry. In some embodiments, structures created by IRIS are preserved over a month of refrigerated storage, suggesting that the femtosecond laser-induced modifications involve relatively long-term molecular/structural alterations that persist for more than a month.

In some embodiments, prior to using the system 300 to modify a refractive index of a sample, a wavefront sensing device is used to measure one or more optical aberrations of a subject's eyes. Based on the measured wavefronts detected by the wavefront sensing device, the one or more optical aberrations are identified and quantified. In some embodiments, the optical aberrations include higher order optical aberrations.

In some embodiments, the computer 318 computes a vision correction profile to correct for the measured aberrations. The vision correction pattern includes refractive index features. A center of the vision correction pattern (e.g., a center of a pattern of the refractive index features) is offset with respect to a center of a pupil of the eye 408 (e.g., centered with respect to a corneal vertex of the eye). How the corneal vertex of the eye is located, measured, and determined, and details regarding the offsetting of the vision correction pattern are disclosed in co-pending U.S. patent application Ser. No: 16/558,298, entitled “Devices and Methods for Measurement And Correction of High-order Optical Aberrations for an Eye Wearing a Contact Lens”, filed on Sep. 2, 2019, the content of which is hereby incorporated by reference in its entirety.

In some embodiments, the sample includes a contact lens. In some embodiments, the sample includes an intraocular lens. In some embodiments, the sample includes a corneal inlay not yet surgically implanted in the subject. In some embodiments, IRIS modifies a refractive index of sample (e.g., contact lens, intraocular lens, corneal inlay) so that an optical aberration of the subject is compensated when the subject places the IRIS-modified sample adjacent to (e.g., contact lens) or into (e.g., corneal inlay, intraocular lens) the subject's eye.

In some embodiments, the system 300 irradiates an optical, polymeric material of the sample with a laser to modify the refractive index in select regions of the material to form structures. The select regions are defined by the focal spot of the laser.

In some embodiments, the structure includes a Bragg grating. In some embodiments, the structure includes a microlens array.

In some embodiments, the structure includes a zone plate or phase plate. In some embodiments, the zone plate includes a pattern of concentrically arranged annular zones having a step height between zones. In some embodiments, an optical height of the steps (e.g., physical height of the step multiplied by a difference between the refractive index of the material and the refractive index of the surrounding media (e.g., air having a refractive index of 1) between the individual zones is one-half that of light (e.g., at a design wavelength). In some embodiments, the zone plate splits a portion of light at the design wavelength equally into the zeroth (un-diffracted) and first diffraction orders. In some embodiments, the zone plate produces a bifocal lens in which (1) the zeroth diffraction order creates a first focus for distant vision and (2) the first diffraction order creates a second focus for near or intermediate vision. In some embodiments, the zone plate corrects (e.g., reduces) overall chromatic aberration at the near vision focus by introducing (diffractive) chromatic dispersion opposite in sign to those chromatic aberrations produced by refraction.

In some embodiments, the zone plate modifies an amount of light in the near and distant foci, depending on a size of the pupil of the subject. In some embodiments, the zone plate increases an amount of light in the distant focus as the pupil size increases. In some embodiments, increasing an amount of light for distance vision includes restricting the zone plate to a core (e.g., central) portion of the lens and to make the outer region of the lens refractive only. In some embodiments, a diffractive lens includes an apodization zone in which the step height between zones in the transition region is progressively reduced. In some embodiments, steps between zones of the zone plate are centered on a base curve to avoid sharp discontinuities in the resulting wavefront and reduce parasitic diffractive effects.

In some embodiments, there is a step circumferential spacing between zones. In some embodiments, there is a step surface profile between zones. In some embodiments, a zone plate maintains a predefined phase relationship of light passing through the zones.

In some embodiments, the structure includes a Fresnel lens.

In some embodiments, IRIS produces ophthalmic lenses, such as intraocular lenses, that have at least two annular zone lenses and an optical step between the annular zone lenses. In some embodiments, IRIS produces a different multifocal diffractive lens having a number of annular lens zones of equal areas, so-called Fresnel zones. In some embodiments, between common border of adjacent Fresnel zones are steps that have an optical height of +λ/2 or −λ/2, where λ is the design wavelength (e.g., for constructive interference of light waves in the zeroth and +1 diffractive order, or the zeroth and −1 diffractive order, respectively). In some embodiments, IRIS produces diffractive lenses having steps between adjacent zones that are odd integer half of the design wavelength. In some embodiments, IRIS produces designs with zones having both +λ/2-steps and −λ/2-steps between subsequent adjacent zones so that constructive interference occurs predominantly in the −1 and/or +1 diffractive order.

In some embodiments, the structure includes a refractive element having a wavefront cross-section phase profile that compensates one or more optical aberrations in the subject's eye. Given the resolution of the femtosecond laser micromachining of about 1 μm, the methods and devices described herein are useful to complementing or replacing current customized wavefront correction methods.

In some embodiments, light pulses from the laser 302 are focused 100 μm below the sample surface using a 60×0.70 NA Olympus LUCPlanFLN microscope objective with an adjustable working distance of 1.5-2.2 mm.

In some embodiments, refractive structures are formed proximate to a top anterior surface (e.g., the surface of the lens that faces the anterior chamber of a human eye) of an intraocular lens. In some embodiments, a positive or negative lens element (three-dimensional) is formed within a 300 μm³ volume, or within a 100 μm³ volume, from the anterior surface of the lens.

In some embodiments, a cylindrical lens structure with a one-dimensional quadratic gradient index is written in an optical polymeric material using three GRIN layers, each 5 μm thick, spaced by approximately 10 μm in the z-direction (e.g., a layer of non-modified optical material having a thickness of about 5 μm to 7 μm is sandwiched between each two adjacent GRIN layers).

In some embodiments, a photosensitizer is used. The photosensitizer includes a chromophore in which there is little or no intrinsic linear absorption in the spectral range of 600-1100 nm. In some embodiments, the photosensitizer enhances the photoefficiency of the two-photon absorption in the formation of refractive structures in the optical, hydrogel polymeric material. In some embodiments, photosensitizers include sodium fluoroscein, coumarin, riboflavin or various UV-blockers, such as UVAM or methine dyes, other UV dyes used in contact lenses or IOLs, and acetaminophen. The photosensitizer may include a chromophore having a two-photon, absorption cross-section of at least 10 GM between a laser wavelength range of 750 nm to 1100 nm.

In IRIS, short laser pulses (e.g., femtosecond laser pulses) having sufficient peak intensities within the focal volume cause a nonlinear absorption of photons (typically multi-photon absorption) that leads to a change in the refractive index of the material within the focal volume. Material just outside of the focal volume is only minimally affected by the laser light. In some embodiments, such femtosecond lasers operate at a high repetition-rate, e.g., 10 MHz, 50 MHz, 80 MHz or higher. In some embodiments, a thermal diffusion time (>0.1 μs) is much longer than the time interval between adjacent laser pulses (on the order of tens of ns). In some embodiments, absorbed laser energy accumulates within the focal volume and increases the local temperature to form laser-induced refractive structures in optical, polymeric materials.

In some embodiments, the presence of water in the polymeric material enhances the formation of the refractive structures. In some embodiments, optical hydrogel polymers have greater processing flexibility in the formation of the refractive structures than zero or low water content optical polymers (e.g., hydrophobic acrylates or low-water (1% to 5% water content) acrylate materials). In some embodiments, there is little to no scattering loss in laser-irradiated regions. In some embodiments, the virtually transparent resulting refractive structures that form in the focal volume are not clearly visible under appropriate magnification without phase contrast enhancement. In some embodiments, an optical material is a polymeric material that transmits at least 80% of visible light (e.g., an optical material does not appreciably scatter or block visible light).

In some embodiments, the region of the optical element that is modified by the focused laser pulse is positioned in the optical element to intersect a second light that impinges on a retina of the subject's eye when the optical element is positioned adjacent to the subject's eye.

### Biological Tissues

In some embodiments, an optical aberration in a subject's eye is corrected by Intra-tissue Refractive Index Shaping (IRIS), which involves irradiating a biological tissue of the subject with light. An irradiated portion of the biological tissue experiences a change in refractive index relative to non-irradiated portions of the biological tissue. In some embodiments, the change results in an increase of the refractive index in the irradiated portion. In some embodiments, refractive structures are formed in the biological tissue by irradiating the biological tissue with light. In some embodiments, refractive structures are formed in non-biological material that is implanted into a patient, for example, a corneal inlay. In some embodiments, the subject is a vertebrate. In some embodiments, the subject is a human (e.g., a human with higher order aberrations). In some embodiments, the subject is a non-human animal (e.g., a cat or a dog).

FIG. 4 is a schematic diagram of a system 400 used to correct optical aberrations in a subject's eye 408, in accordance with some embodiments. The eye 408 includes a retina 410, a cornea 412, and a lens 418.

The system 400 includes a beam steering unit 402. In some embodiments, the beam steering unit 402 includes translation stages, identical or similar to those in sample manipulation unit 314. The beam steering unit 402 directs the laser beam to specific locations of the subject's eye 408. In some embodiments, the beam steering unit 402 also includes focusing optics to deliver light to a specific focal location in the eye 408. In some embodiments, the beam steering unit allows the focusing optics to vary a position of the focal location along a depth dimension.

The system 400 includes a beamsplitter 404 that directs (e.g., transmits) a portion of the laser light toward the eye 408. The beamsplitter 404 also direct measurement light from a wavefront sensing unit 406 toward the eye 408.

In some embodiments, the correction process includes: (1) using the wavefront sensing unit 406 to detect and measure lower (e.g., prism) and higher order (e.g., trefoil, coma, spherical aberration) aberrations along the optical path of a given eye, (2) calculating the topography and magnitude of refractive index changes required to achieve the necessary aberration correction, and aligning the topography of refractive index changes with respect to a corneal vertex of the subject's eye, and (3) focusing the femtosecond laser pulses either into the cornea or intraocular lens in order to carry out the micromachining necessary to induce the required refractive index change.

To detect wavefront aberration from the eye 408 using the wavefront sensing unit, the shutter 312 is closed so that no laser light from laser 302 impinges on the eye 408. The wavefront sensing unit 406 includes a light source that directs light traveling in a direction indicated by an arrow 414 toward the eye 408. Light 416 is reflected from the retina 410 of the eye 408, and includes distortions caused by one or more optical aberrations in the eye 408. A wavefront sensor in the wavefront sensing unit 406 detects the light 416 and computes, either at the sensing unit 406 or the computer 318, the optical aberrations present in the eye 408, including higher order aberrations. In some embodiments, the wavefront sensing unit 406 includes an array of lenses arranged to focus incoming light onto multiple spots. In some embodiments, the wavefront sensing unit 406 includes a Shack-Hartmann wavefront sensor. In some embodiments, the wavefront sensing unit 406 includes a Hartmann array, which is a plate with an array of apertures (e.g., through-holes) defined therein. In some embodiments, the wavefront sensing unit 406 includes a wavefront sensor described in U.S. Pat. No. 9,427,156, entitled “Devices and Methods for Wavefront Sensing and Corneal Topography” or a wavefront sensor described in U.S. Patent Publication No US 2018/0160899, entitled “Devices and Methods for Refractive Power Measurements of an Eye with Reduced Errors,” both of which are incorporated by reference herein.

The higher order aberrations measured by the wavefront sensing unit 406 are identified and quantified by the computer 318 and/or the wavefront sensing unit 406. The higher-order aberrations are corrected using a vision correction pattern formed by locally changing a refractive index in the cornea 412 or the lens 418. The vision correction pattern is calculated by the computer 318 and includes information about the position and shape of the structures to be written into the polymeric material and/or the biological tissue to compensate for those aberrations or to provide vision correction to the patient. The computer 318 communicates with the other components of the system 300 or the system 400 to direct laser light to irradiate one or more regions of the polymeric material and/or the biological tissue. The irradiated region provides a wavefront cross-section phase profile that includes one or more desired refractive features to provide desired vision correction. In some embodiments, the laser light includes pulsed laser light. The energy of the light pulses ranges from 0.05 nJ to 1000 nJ.

The vision correction pattern includes refractive index features. A center of the vision correction pattern (e.g., a center of a pattern of the refractive index features) is offset with respect to a center of a pupil of the eye 408. The center of the vision correction pattern is centered with respect to a corneal vertex of the eye. How the corneal vertex of the eye is measured and determined, and other details regarding the offsetting of the vision correction pattern are disclosed in co-pending U.S. patent application Ser. No.: 16/558,298, entitled “Devices and Methods for Measurement And Correction of High-order Optical Aberrations for an Eye Wearing a Contact Lens”, filed on Sep. 2, 2019, the content of which is hereby incorporated by reference in its entirety.

In some embodiments, after the vision correction pattern is calculated by the computer 318 (including an offset (with respect to a center of a pupil of the subject's eye) to center the correction pattern about the corneal vertex of the subject's eye), the computer 318 provides control signals to the beam steering unit 402 so that a focal region of the laser beam is directed to a correct portion of the eye 408, in accordance to the vision correction pattern. The system 400 corrects the optical aberrations of the eye 408 by opening the shutter 312, and light pulses from the laser 302 correct optical aberrations of the eye 408 by locally changing a refractive index in the cornea 412 or the lens 418. A pattern of irradiation of the laser light follows the vision correction pattern calculated by the computer 318 to correct the higher order aberrations measured by the wavefront sensing unit 406. After the eye 408 is irradiated with laser pulses, the shutter 312 is closed, and the wavefront sensing unit 406 directs measurement light toward the eye 408 to measure a wavefront that reflects off the retina 410 of the eye 408.

Once the micromachining is complete, the wavefront sensing unit 406 makes a new measurement on the subject's eye 408. The shutter 312 is closed to allow the wavefront sensing unit 406 to direct light from its light source (indicated by the arrow 414) toward the eye 408. Light reflects from the retina 410 of the eye 408, and experiences the phase profile introduced by the corrections made by IRIS, reducing (e.g., eliminating) one or more optical aberrations previously present in the eye 408.

In some embodiments, a resolution of the femtosecond laser micromachining is about 1 μm, and the noninvasive method described herein is used to complement current customized wavefront correction methods. In some embodiments, the noninvasive method described herein is used instead of other current customized wavefront correction methods.

In some embodiments, a multiple-photon-absorbing chromophore is applied to a biological tissue prior to modifying the refractive index of the tissue. In some embodiments, the multiple-photon-absorbing chromophore is a two-photon-absorbing chromophore. In some embodiments, the biological tissue is a tissue of a lens (e.g., a crystalline lens in the subject's eye). In some embodiments, the biological tissue is an ocular tissue that includes tissues of a cornea. In some embodiments, IRIS does not change a surface of the biological tissue, and reduces or eliminates regrowth of tissue cells. In some embodiments, IRIS provides a relatively small focus spot (e.g., approximately 1 micrometer diameter) and allows finer correction to be made to the biological tissue.

In some embodiments, IRIS is induced using 0.3 nJ and 0.5 nJ pulses (from lasers having an average laser power of 30 mW and 45 mW), without any photo-disruption or tissue destruction of the biological tissue. In some embodiments, a 0.70 NA long-working-distance objective is used and average laser power breakdown thresholds for cornea and lens are approximately 55 mW and 75 mW, respectively (e.g., corresponding to pulse energies of 0.6 nJ and 0.8 nJ, respectively).

In some embodiments, a focal region of the biological tissue that is modified by the laser is positioned in the biological tissue to intersect a second light that impinges on a retina of the subject's eye.

In some embodiments, IRIS creates a diffractive multifocal pattern to provide near, intermediate and/or far foci or to increase a subject's depth of focus. In some embodiments, IRIS creates a Fresnel lens pattern having a half wave phase change. In some embodiments, IRIS creates a refractive multifocal. In some embodiments, a refractive multifocal includes multiple concentric rings of differing refractive power or segments of differing refractive power. In some embodiments, IRIS creates different multifocals in combination with a binocular modified monovision presbyopia correction.

In some embodiments, IRIS provides vision corrections via corneal presbyopia treatments in the cornea using multifocal diffraction patterns. In some embodiments, because laser induced refractive index change via IRIS does not require incisions, no significant alteration from corneal or epithelial re-modeling and healing is triggered.

In some embodiments, IRIS creates a vision correction profile that is offset with respect to a center of a pupil of a subject's eye, the vision correction profile having a refractive index profile that places myopic hyperopic defocus on a periphery of the retina of a subject's eye to prevent or slow myopia progression in the subject. In some embodiments, IRIS modifies a refractive index of a polymer in an optical device (e.g., intraocular lens, contact lens, or corneal inlay) or an ocular tissue of an eye of the subject, by irradiating select regions of the polymer or ocular tissue, respectively, with a focused, visible or near-IR laser below the optical breakdown threshold of the material. In some embodiments, the optical structures exhibit a change in refractive index, and have little or no scattering loss. In some embodiments, IRIS does not ablate or remove biological tissue or polymer material from the irradiated region. In some embodiments, IRIS forms a multifocal region that includes peripheral zone having a first dioptric power and a core zone. The core zone has a second dioptric power different from the first dioptric power. In some embodiments, the core zone is surrounded by the peripheral zone. In some embodiments, the multifocal region is formed in a cornea or natural crystalline lens. In some embodiments, the multifocal region is formed in an intraocular lens or optical device.

In some embodiments, IRIS creates a refractive structure with a gradient index in one, two or three dimensions of the optical material. In some embodiments, the gradient refractive structure is formed by continuously scanning a continuous stream of femtosecond laser pulses having a controlled focal volume in and along at least one contiguous segment (scan line) in the optical material while varying the scan speed and/or the average laser power. Such variations create a gradient refractive index in the polymer along the segment.

In some embodiments, rather than creating discrete, individual, or even grouped or clustered, adjoining segments of refractive structures with a constant change in the index of refraction in the material, a gradient refractive index is created within the refractive structure, and thereby in the optical material, by continuously scanning a continuous stream of pulses.

In some embodiments, the refractive modification in the material arises from a multiphoton absorption process. In some embodiments, a well-controlled focal volume corrected for spherical (and other) aberrations provides a segment having consistent and constant depth over the length of the scan.

In some embodiments, multiple segments are written into the material in a layer using different scan speeds and/or different average laser power levels for various segments to create a gradient index profile across the layer, e.g., transverse to the scan direction.

In some embodiments, a laser-modified GRIN layer includes a number of adjacent refractive segments having a change in the index of refraction in relation to the index of refraction of non-modified polymeric material formed with continuous streams of light pulses from a laser contiguously scanned along regions of the polymeric material.

In some embodiments, each of the number of adjacent refractive segments has an independent line width. An intersegment spacing of two adjacent refractive segments is less than an average line width of the two adjacent segments so that there is overlap of the adjacent segments, and the GRIN layer is characterized by a variation in index of refraction in a direction of at least one of: (i) a transverse cross section of the adjacent refractive segments; and (ii) a lateral cross section of the refractive segments.

In some embodiments, multiple, spaced gradient index (GRIN) layers are written into the material along the z-direction (e.g., generally the light propagation direction through the material) to provide a desired refractive change in the material that corrects for some, most, or all higher order aberrations of a patient's eye.

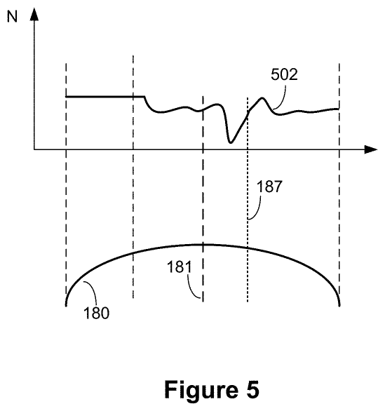

FIG. 5 is a schematic diagram illustrating a refractive index profile 502 of a lens (e.g., a contact lens, an intraocular lens, etc.) in accordance with some embodiments. The refractive index of the contact lens varies across the surface of the contact lens to compensate for higher order aberrations in a patient's eye. As shown in FIG. 5, the center of the refractive index profile is offset from the centerline 181 of the contact lens 180 (e.g., for alignment with the visual axis 187 of the eye).

As shown in FIG. 5, in some embodiments, the surface profile of the contact lens 180 is similar to a surface profile of a conventional contact lens. In some embodiments, the surface profile of the contact lens 180 includes a surface profile configured to compensate for one or more of the higher order aberrations (e.g., a superposition of the surface profile 188 shown in FIG. 1C). In some cases, combining the physical variation in the surface profile (e.g., a thickness, a curvature, etc.) with the refractive index profile allows correction of even higher order aberrations, or correction of (or compensation for) any errors induced by machining or any variation in the lens material. In some embodiments, the contact lens 180 is a multifocal lens, as shown in FIG. 1D.

In light of these principles, we turn to certain embodiments.

In accordance with some embodiments, a method includes providing a first light to a first region of an optical element, thereby modifying a refractive index profile in the first region of the optical element, while offsetting a center of the first region with respect to a center of the optical element. The refractive index profile is configured to compensate for an optical aberration of a subject's eye.

In some embodiments, a refractive index profile is a vision correction profile. In some embodiments, the optical aberration includes higher-order aberrations. In some embodiments, the first region includes a multifocal region. The multifocal region includes a peripheral zone having a first dioptric power and a core zone. The core zone has a second dioptric power different from the first dioptric power.

In some embodiments, the core zone is surrounded by the peripheral zone. In some embodiments, providing the first light to the first region of the optical element causes modification of a refractive index at a first location of the optical element from a first value of the refractive index to a second value of the refractive index that is different from the first value. In some embodiments, the first value is an original value (e.g., not modified by laser irradiation). In some embodiments, a difference between the first value and the second value is greater than 0.005.

In some embodiments, the optical element includes a contact lens. In some embodiments, the contact lens is configured for placement adjacent to the subject's eye. In some embodiments, the optical element includes an intraocular lens. In some embodiments, the intraocular lens is configured for implant in the subject's eye. In some embodiments, the optical element includes a corneal inlay in the subject's eye (or a corneal inlay for implant in the subject's eye).

In some embodiments, the optical element includes a polymer. In some embodiments, the polymer includes a hydrogel. In some embodiments, the refractive index profile includes a gradient refractive index formed by scanning the first light along a contiguous segment in the optical material while varying a scan speed of the first light.

In some embodiments, the refractive index profile includes a gradient refractive index formed by scanning the first light along a contiguous segment in the optical material while varying an average power of the first light.

In some embodiments, the refractive index profile includes a gradient refractive index formed by scanning the first light along a contiguous segment in the optical material while varying a peak intensity of the first light. In some embodiments, the peak intensity of the first light is varied while maintaining the average power of the first light.

In some embodiments, the first region is positioned in the optical element to intersect a second light that impinges on a retina of the subject's eye when the optical element is positioned adjacent to the subject's eye. In some embodiments, the refractive index profile includes multiple gradient index layers defined within the optical element so that the second light intersects each gradient index layer of the multiple gradient index layers when the optical element is positioned adjacent to the subject's eye. In some embodiments, the multiple gradient index layers are arranged along a direction that is non-perpendicular to a propagation direction of the second light. In some embodiments, the multiple gradient index layers are arranged along a direction substantially parallel (e.g., having an angle less than 10 degrees, less than 5 degrees, or less than 3 degrees) to a propagation direction of the second light.

In some embodiments, the refractive index profile includes diffractive structures to provide two or more foci for the second light. In some embodiments, the refractive index profile creates near, intermediate and/or far foci, or increases an eye's depth of focus. In some embodiments, the refractive index profile includes a Fresnel lens pattern having a half wave phase change.

In some embodiments, the first light is provided in pulses, and a temporal separation between the pulses of the first light is shorter than a thermal diffusion time in the optical element. In some embodiments, the first light is provided in pulses, and a temporal separation between the pulses of the first light is shorter than a thermal diffusion time in a biological tissue.

In some embodiments, the optical element includes a lens located in situ. In some embodiments, the optical element includes a lens located ex vivo.

In some embodiments, offsetting the center of the first region comprises centering the first region with respect to a corneal vertex of the subject's eye. In some embodiments, the corneal vertex is determined based on subject-fixated coaxially sighted light reflex. In some embodiments, an optical element is made by the method described above.

In accordance to some embodiments, a method includes providing a first light to a first region of a biological tissue in a subject's eye, thereby modifying a refractive index profile in the first region of the biological tissue, while offsetting a center of the first region with respect to a center of a pupil of the subject's eye. The refractive index profile is configured to correct an optical aberration of the subject's eye.

In some embodiments, the biological tissue includes a natural crystalline lens. In some embodiments, the optical aberration includes higher-order aberrations. In some embodiments, the first region includes a multifocal region. In some embodiments, the first light has a wavelength between about 350 nm to about 600 nm.

In some embodiments, the method further includes controlling an intensity of the first light to be lower than a damage threshold of the biological tissue. In some embodiments, providing the first light to the first region of the biological tissue causes modification of a refractive index at a first location of the biological tissue from a first value of the refractive index to a second value of the refractive index that is different from the first value.

In some embodiments, the first value is higher than the original value. In some embodiments, the first value is lower than the original value. In some embodiments, whether the first value is lower or higher than the original value depends on a combination of materials and wavelengths used. In some embodiments, a difference between the first value and the second value is greater than 0.005. In some embodiments, the first location corresponds to a focal region of the first light, a second location is located outside the focal region of the first light, and a refractive index at the second location is not modified.

In some embodiments, the refractive index profile is created by scanning the first light through a volume of the biological tissue. In some embodiments, the first light includes femtosecond laser pulses. In some embodiments, the refractive index profile causes less than 0.1% (e.g., or some other value) of scattering loss and an optical clarity of the biological tissue is preserved. In some embodiments, the refractive index profile of the first region in the biological tissue is a subsurface refractive index profile.

In some embodiments, the refractive index profile includes features that place myopic defocus on a periphery of the retina. In some embodiments, offsetting the center of the first region includes centering the first region with respect to a corneal vertex of the subject's eye. In some embodiments, the corneal vertex is determined based on subject-fixated coaxially sighted light reflex.

In accordance to some embodiments, a method of improving vision of a subject includes placing an optical element having a vision correction profile in an optical path of light that impinges on a retina of the subject. A center of the vision correction profile is offset from a center of the optical element.

In some embodiments, placing the optical element having the vision correction profile includes adding the vision correction profile to the optical element. In some embodiments, placing the optical element having the vision correction profile includes adding the vision correction profile to the optical element in situ.

In some embodiments, the vision correction profile includes a refractive index profile formed by using pulses of a first light to modify a refractive index of the optical element at a first location. In some embodiments, the optical element includes a Prosthetic Replacement of the Ocular Surface Ecosystem (e.g. PROSE device) and improving vision includes reducing higher-order aberrations.

In some embodiments, improving vision includes one or more of: improving high contrast visual acuity, improving low contrast visual acuity, improving subjective visual quality, or decreasing a root-mean square of higher-order wavefront aberrations. In some embodiments, the vision correction profile includes a surface profile of the optical element. In some embodiments, the surface profile of the optical element is formed by removing material from the optical element. In some embodiments, the vision correction profile further includes modifying a refractive index at a first location of the surface profile using pulses of a first light. In some embodiments, the first location is subsurface. In some embodiments, multi-photon absorption of the first light occurs at the first location. In some embodiments, the center of the vision correction profile is centered with respect to a corneal vertex of the subject's eye. In some embodiments, the corneal vertex is determined based on subject-fixated coaxially sighted light reflex.

In accordance to some embodiments, a method includes improving vision of a subject by placing an optical element having a vision correction profile in an optical path of light that impinges on a retina of the subject. A center of the vision correction profile is offset from a center of the optical element.

In some embodiments, the center of the vision correction profile is centered with respect to a corneal vertex of the subject's eye. In some embodiments, the corneal vertex is determined based on subject-fixated coaxially sighted light reflex.

In accordance to some embodiments, a lens includes a substrate with a refractive index profile, the refractive index profile having a first refractive index at a first location of the substrate, and a second refractive index, different from the first refractive index, at a second location of the substrate, wherein a center of the refractive index profile is offset from a center of the lens.

In some embodiments, a material of the substrate at the first location and a material of the substrate at the second location are a same material, the material of the substrate at the first location having been exposed to femtosecond laser pulses so that a refractive index of the material of the substrate at the first location is different from a refractive index of the material of the substrate at the second location.

In some embodiments, the first location is at a distance of about 1 μm from the second location. In some embodiments, the center of the refractive index profile is centered with respect to a corneal vertex of a subject's eye. In some embodiments, the corneal vertex is determined based on subject-fixated coaxially sighted light reflex.

Some embodiments may be described with respect to the following clauses.

Clause 1. A method, comprising:
providing a first light to a first region of an optical element, thereby modifying a refractive index profile in the first region of the optical element, while offsetting a center of the first region with respect to a center of the optical element, wherein the refractive index profile is configured to compensate for an optical aberration of a subject's eye.

Clause 2. The method of clause 1, wherein the optical aberration comprises higher-order aberrations.

Clause 3. The method of clause 1 or 2, wherein the first region comprises a multifocal region.

Clause 4. The method of clause 3, wherein the multifocal region comprises a peripheral zone having a first dioptric power and a core zone, the core zone having a second dioptric power different from the first dioptric power.

Clause 5. The method of any of clauses 1-4, wherein providing the first light to the first region of the optical element causes modification of a refractive index at a first location of the optical element from a first value of the refractive index to a second value of the refractive index that is different from the first value.

Clause 6. The method of clause 5, wherein a difference between the first value and the second value is greater than 0.005.

Clause 7. The method of any of clauses 1-6, where the optical element comprises a contact lens.

Clause 8. The method of any of clauses 1-6, wherein the optical element comprises an intraocular lens.

Clause 9. The method of any of clauses 1-6, wherein the optical element comprises a corneal inlay.

Clause 10. The method of any of clauses 1-9, wherein the optical element comprises a polymer.

Clause 11. The method of clause 10, wherein the polymer comprises a hydrogel.

Clause 12. The method of any of clauses 1-11, wherein the refractive index profile comprises a gradient refractive index formed by scanning the first light along a contiguous segment in the optical material while varying a scan speed of the first light.

Clause 13. The method of any of clauses 1-12, wherein the refractive index profile comprises a gradient refractive index formed by scanning the first light along a contiguous segment in the optical material while varying an average power of the first light.

Clause 14. The method of any of clauses 1-13, wherein the refractive index profile comprises a gradient refractive index formed by scanning the first light along a contiguous segment in the optical material while varying a peak intensity of the first light.

Clause 15. The method of any of clauses 1-14, wherein the first region is positioned in the optical element to intersect a second light that impinges on a retina of the subject's eye when the optical element is positioned adjacent to the subject's eye, and the refractive index profile comprises multiple gradient index layers defined within the optical element so that the second light intersects each gradient index layer of the multiple gradient index layers when the optical element is positioned adjacent to the subject's eye.

Clause 16. The method of any of clauses 1-15, wherein the refractive index profile comprises diffractive structures to provide two or more foci for the second light.

Clause 17. The method of any of clauses 1-16, wherein the first light is provided in pulses, and a temporal separation between the pulses of the first light is shorter than a thermal diffusion time in the optical element.

Clause 18. The method of any of clauses 1-17, wherein the optical element comprises a lens located in situ.

Clause 19. The method of any of clauses 1-18, wherein the optical element comprises a lens located ex vivo.

Clause 20. The method of any of clauses 1-19, wherein offsetting the center of the first region comprises centering the first region with respect to a corneal vertex of the subject's eye.

Clause 21. The method of clause 20, wherein the corneal vertex is determined based on subject-fixated coaxially sighted light reflex.

Clause 22. An optical element made by the method of any of clauses 1-21.

Clause 23. A method, comprising:
providing a first light to a first region of a biological tissue in a subject's eye, thereby modifying a refractive index profile in the first region of the biological tissue, while offsetting a center of the first region with respect to a center of a pupil of the subject's eye, wherein the refractive index profile is configured to correct an optical aberration of the subject's eye.

Clause 24. The method of clause 23, wherein the biological tissue comprises a natural crystalline lens.

Clause 25. The method of clause 23 or 24, wherein the optical aberration comprises higher-order aberrations.

Clause 26. The method of any of clauses 23-25, wherein the first region comprises a multifocal region.

Clause 27. The method of any of clauses 23-26, wherein the first light has a wavelength between about 350 nm to about 600 nm.

Clause 28. The method of any of clauses 23-27, further comprising controlling an intensity of the first light to be lower than a damage threshold of the biological tissue.

Clause 29. The method of any of clauses 23-28, wherein providing the first light to the first region of the biological tissue causes modification of a refractive index at a first location of the biological tissue from a first value of the refractive index to a second value of the refractive index that is different from the first value.

Clause 30. The method of clause 29, wherein the first value is higher than the original value.

Clause 31. The method of clause 29, wherein the first value is lower than the original value.

Clause 32. The method of any of clauses 29-31, wherein a difference between the first value and the second value is greater than 0.005.

Clause 33. The method of any of clauses 29-32, wherein:
the first location corresponds to a focal region of the first light;
a second location is located outside the focal region of the first light; and
a refractive index at the second location is not modified.

Clause 34. The method of any of clauses 23-33, wherein the refractive index profile is created by scanning the first light through a volume of the biological tissue.

Clause 35. The method of any of clauses 23-34, wherein the first light comprise femtosecond laser pulses.

Clause 36. The method of any of clauses 23-35, wherein the refractive index profile causes less than 0.1% of scattering loss and an optical clarity of the biological tissue is preserved.

Clause 37. The method of any of clauses 23-36, wherein the refractive index profile of the first region in the biological tissue comprises a subsurface refractive index profile.

Clause 38. The method of any of clauses 23-37, wherein the refractive index profile comprises features that place myopic defocus on a periphery of the retina.

Clause 39. The method of any of clauses 23-38, wherein offsetting the center of the first region comprises centering the first region with respect to a corneal vertex of the subject's eye.

Clause 40. The method of clause 39, wherein the corneal vertex is determined based on subject-fixated coaxially sighted light reflex.

Clause 41. A method of improving vision of a subject, comprising:
placing an optical element having a vision correction profile in an optical path of light that impinges on a retina of the subject, wherein a center of the vision correction profile is offset from a center of the optical element.

Clause 42. The method of clause 41, wherein placing the optical element having the vision correction profile includes adding the vision correction profile to the optical element.

Clause 43. The method of clause 41 or 42, wherein the vision correction profile comprises a refractive index profile formed by using pulses of a first light to modify a refractive index of the optical element at a first location.

Clause 44. The method of any of clauses 41-43, wherein the optical element comprises a scleral lens and improving vision comprises reducing higher-order aberrations.

Clause 45. The method of any of clauses 41-44, wherein improving vision comprises one or more of: improving high contrast visual acuity, improving low contrast visual acuity, improving subjective visual quality, or decreasing a root-mean square of higher-order wavefront aberrations.

Clause 46. The method of any of clauses 41-45, wherein the vision correction profile comprises a surface profile of the optical element.

Clause 47. The method of clause 46, wherein the surface profile of the optical element is formed by removing material from the optical element.

Clause 48. The method of clause 46 or 47, wherein the vision correction profile further comprises modifying a refractive index at a first location of the surface profile using pulses of a first light.

Clause 49. The method of clause 48, wherein the first location is subsurface.

Clause 50. The method of clause 48 or 49, wherein multi-photon absorption of the first light occurs at the first location.

Clause 51. The method of any of clauses 41-50, wherein the center of the vision correction profile is centered with respect to a corneal vertex of the subject's eye.

Clause 52. The method of clause 51, wherein the corneal vertex is determined based on subject-fixated coaxially sighted light reflex.

Clause 53. A method, comprising:
improving vision of a subject by placing an optical element having a vision correction profile in an optical path of light that impinges on a retina of the subject, wherein a center of the vision correction profile is offset from a center of the optical element.

Clause 54. The method of clause 53, wherein the center of the vision correction profile is centered with respect to a corneal vertex of the subject's eye.

Clause 55. The method of clause 54, wherein the corneal vertex is determined based on subject-fixated coaxially sighted light reflex.

Clause 56. A lens, comprising:
a substrate with a refractive index profile, the refractive index profile having a first refractive index at a first location of the substrate, and a second refractive index, different from the first refractive index, at a second location of the substrate, wherein a center of the refractive index profile is offset from a center of the lens.

Clause 57. The lens of clause 56, wherein a material of the substrate at the first location and a material of the substrate at the second location are a same material, the material of the substrate at the first location having been exposed to femtosecond laser pulses so that a refractive index of the material of the substrate at the first location is different from a refractive index of the material of the substrate at the second location.

Clause 58. The lens of clause 56 or 57, wherein the first location is at a distance of about 1 μm from the second location.

Clause 59. The lens of any of clauses 56-58, wherein the center of the refractive index profile is centered with respect to a corneal vertex of a subject's eye.

Clause 60. The lens of clause 59, wherein the corneal vertex is determined based on subject-fixated coaxially sighted light reflex.

The foregoing description, for purpose of explanation, has been described with reference to specific embodiments. However, the illustrative discussions above are not intended to be exhaustive or to limit the scope of claims to the precise forms disclosed. Many modifications and variations are possible in view of the above teachings. For example, the methods described above may be used for designing and making lenses for spectacles (e.g., eyeglasses). The embodiments were chosen and described in order to best explain the principles of the various described embodiments and their practical applications, to thereby enable others skilled in the art to best utilize the invention and the various described embodiments with various modifications as are suited to the particular use contemplated.

What is claimed is:

1. A method, comprising:
providing a first light to a first region of an optical element, thereby modifying a refractive index profile in the first region of the optical element, while offsetting a center of the first region with respect to a center of the optical element, wherein the refractive index profile is configured to compensate for an optical aberration of a subject's eye.

2. The method of claim 1, wherein the optical aberration comprises higher-order aberrations.

3. The method of claim 1, wherein the first region comprises a multifocal region.

4. The method of claim 3, wherein the multifocal region comprises a peripheral zone having a first dioptric power and a core zone, the core zone having a second dioptric power different from the first dioptric power.

5. The method of claim 1, wherein providing the first light to the first region of the optical element causes modification of a refractive index at a first location of the optical element from a first value of the refractive index to a second value of the refractive index that is different from the first value.

6. The method of claim 5, wherein a difference between the first value and the second value is greater than 0.005.

7. The method of claim 1, where the optical element comprises a contact lens, an intraocular lens, or a corneal inlay.

8. The method of claim 1, wherein the optical element comprises a polymer.

9. The method of claim 8, wherein the polymer comprises a hydrogel.

10. The method of claim 1, wherein the refractive index profile comprises a gradient refractive index formed by scanning the first light along a contiguous segment in the optical material while varying at least one of: a scan speed of the first light, an average power of the first light, or a peak intensity of the first light.

11. The method of claim 1, wherein the first region is positioned in the optical element to intersect a second light that impinges on a retina of the subject's eye when the optical element is positioned adjacent to the subject's eye, and the refractive index profile comprises multiple gradient index layers defined within the optical element so that the second light intersects each gradient index layer of the multiple gradient index layers when the optical element is positioned adjacent to the subject's eye.

12. The method of claim 1, wherein the refractive index profile comprises diffractive structures to provide two or more foci for the second light.

13. The method of claim 1, wherein the first light is provided in pulses, and a temporal separation between the pulses of the first light is shorter than a thermal diffusion time in the optical element.

14. The method of claim 1, wherein offsetting the center of the first region comprises centering the first region with respect to a corneal vertex of the subject's eye.

15. The method of claim 14, wherein the corneal vertex is determined based on subject-fixated coaxially sighted light reflex.

16. An optical element made by the method of claim 1.

17. A lens, comprising:
a substrate with a refractive index profile, the refractive index profile having a first refractive index at a first location of the substrate, and a second refractive index, different from the first refractive index, at a second location of the substrate, wherein a center of the refractive index profile is offset from a center of the lens.

18. The lens of claim 17, wherein a material of the substrate at the first location and a material of the substrate at the second location are a same material, the material of the substrate at the first location having been exposed to femtosecond laser pulses so that a refractive index of the material of the substrate at the first location is different from a refractive index of the material of the substrate at the second location.

19. The lens of claim 17, wherein the first location is at a distance of about 1 μm from the second location.

20. The lens of claim 17, wherein the center of the refractive index profile is centered with respect to a corneal vertex of a subject's eye.
